(19) United States
(12) Patent Application Publication
(10) Pub. No.: US 2026/0253227 A1
(43) Pub. Date: Aug. 27, 2026

(54) METHODS FOR SURGICAL SEGMENT SELECTION IN ADOLESCENT IDIOPAHIC SCOLIOSIS ASSISTED BY DEEP LEARNING

(71) Applicants: NANJING DRUM TOWER HOSPITAL, Nanjing (CN); NANJING UNIVERSITY, Nanjing (CN)

(72) Inventors: Zhong HE, Nanjing (CN); Wujun LI, Nanjing (CN); Neng LU, Nanjing (CN); Zezhang ZHU, Nanjing (CN)

(73) Assignees: NANJING DRUM TOWER HOSPITAL, Nanjing (CN); NANJING UNIVERSITY, Nanjing (CN)

(21) Appl. No.: 19/643,739

(22) Filed: Apr. 9, 2026

Related U.S. Application Data
(63) Continuation-in-part of application No. PCT/CN2024/093582, filed on May 16, 2024.

(30) Foreign Application Priority Data
Oct. 9, 2023 (CN) 202311293757.X

(51) Int. Cl. G06T 7/11; A61B 17/56; A61B 34/10

(52) U.S. Cl. CPC G06T 7/11; A61B 17/56; A61B 34/10; A61B 2017/568; A61B 2034/104; A61B 2034/105; G06T 2207/20081; G06T 2207/30012

(57) ABSTRACT

A method for surgical segment selection in adolescent idiopathic scoliosis (AIS) assisted by deep learning is provided, the method comprises: acquiring a clinical case image, annotating positions and types of a surgical fixation segment and a non-fixated segment in thoracic vertebrae and lumbar vertebrae in the clinical case image, and training a network model; inputting a spinal image of a patient into the trained network model, wherein the network model segments the spinal image according to learned image features, divides the spinal image into different regions, and outputs a predicted type and a confidence for each region; correcting the confidence by a process of position correction.

### CROSS-REFERENCE TO RELATED APPLICATIONS

This application is a Continuation-in-part of International Application No. PCT/CN2024/093582, filed on May 16, 2024, which claims priority to Chinese Patent Application No. 202311293757.X, filed on Oct. 9, 2023, the entire contents of which are incorporated herein by reference.

### TECHNICAL FIELD

The present disclosure generally relates to a field of computer and medical science, and in particular to a method for surgical segment selection in adolescent idiopathic scoliosis (AIS) assisted by deep learning.

### BACKGROUND

The combination of deep learning and medicine is a current hot topic and development direction in the field of technology and health. The deep learning is an artificial intelligence manner that mimics a neural network of a human brain, which can automatically learn and extract useful information from a large amount of data. The medicine is a science related to human life and health, which needs to process various complex medical data and problems. Combining deep learning and medicine can provide doctors with faster, more accurate, and more objective diagnostic references, provide patients with more personalized, efficient, and safe treatment plans, and further provide researchers with more innovative, in-depth, and extensive research approaches. The deep learning has demonstrated its powerful potential and advantages in multiple fields such as medical image analysis, electronic health records, genomics, and drug development.

However, in China, formulation of most surgical strategies still mainly depends on personal experience of surgeons. Differences in distribution of medical resources in different regions may lead to misdiagnosis and mistreatment of diseases. Adolescent idiopathic scoliosis (AIS) is a typical example. The AIS refers to a three-dimensional spinal deformity that occurs in the spine during adolescent growth and development for unknown reasons. The AIS is the most common spinal deformity in adolescents. The AIS, obesity, and myopia are listed as three major diseases that endanger the health of primary and secondary school students in China. Adolescent scoliosis not only affects the appearance and self-confidence of a patient, but also causes damage to systems such as respiration, cardiovascular, and nerves. Adolescent scoliosis may even endanger life. Therefore, timely detection and correct treatment of AIS are crucial. However, selection of a surgical segment for AIS is highly controversial. Although a Lenke classification system has been widely used to guide selection of scoliosis requiring fusion, the Lenke classification system does not clearly indicate a specific fusion segment. For different curve types, scholars from various countries have proposed multiple strategies for fusion segment selection. However, due to a persistently high incidence of long-term complications, consensus has not been reached.

Current existing technologies can screen for scoliosis. The current existing technologies can plan a surgical path for a patient who has already been confirmed to undergo surgery and for whom a surgical segment has been determined. However, these are manners to assist doctors in diagnosis, which do not involve determination of surgery or selection of a surgical position.

Therefore, there is an urgent need for a deep learning-based intelligent selection method for an AIS surgical segment. The method can achieve automatic segmentation of spinal X-ray images, vertebral body classification, and intelligent recommendation of fusion segments. The method can provide objective and accurate surgical decision-making references for clinicians.

### SUMMARY

One or more embodiments of the present disclosure provide a method for surgical segment selection in adolescent idiopathic scoliosis (AIS) assisted by deep learning. The method includes: step 1: acquiring a clinical case image, annotating positions and types of a surgical fixation segment and a non-fixated segment in thoracic vertebrae and lumbar vertebrae in the clinical case image, and training a network model; step 2: inputting a spinal image of a patient into the trained network model, wherein the network model segments the spinal image according to learned image features, divides the spinal image into different regions, and outputs a predicted type and a confidence for each region; and step 3: correcting the confidence by a process of position correction.

The technical solution provided in the present disclosure has following technical effects:
1. The method employs two deep learning frameworks including the UNet learning framework and the YOLOv8 learning framework. The method introduces a Swin Transformer module into the UNet learning framework to improve a feature extraction capability of the UNet learning framework. The structures of the two deep learning frameworks can simultaneously extract semantic features and instance features in the spinal image, thereby improving the accuracy of the deep learning network model.
2. The method employs a process of position correction by automatic calculation and manual correction to adjust and optimize confidence, thereby reducing noise of the network model. The method corrects the confidence according to prior knowledge, so that the confidence better conforms to clinical prior rules for fusion segment selection in AIS surgery. A doctor may select a surgical fusion segment according to the corrected confidence in combination with clinical data, thereby providing a personalized treatment plan for a patient and reducing an incidence of postoperative complications.
3. The method can effectively assist a doctor in preoperative planning for AIS, improve a correction effect and safety, and improve a quality of life of the patient.

### BRIEF DESCRIPTION OF THE DRAWINGS

The present disclosure will be further described by way of exemplary embodiments. These exemplary embodiments will be described in detail with reference to the drawings. These embodiments are not limited. In these embodiments, the same numbers denote the same structures, wherein:

FIG. 1 is a flowchart of an exemplary process for surgical segment selection in AIS assisted by deep learning according to some embodiments of the present disclosure;

FIG. 2 is a schematic diagram of an exemplary process for surgical segment selection in AIS assisted by deep learning according to some embodiments of the present disclosure;

FIG. 3 is a schematic diagram of an exemplary structure of Swin-UNet according to some embodiments of the present disclosure;

FIG. 4 is a schematic diagram of an exemplary multi-scale image feature extraction according to some embodiments of the present disclosure; and

FIG. 5 is an exemplary diagram of a spinal instance segmentation result according to some embodiments of the present disclosure.

### DETAILED DESCRIPTION

The technical solutions in the embodiments of the present disclosure will be clearly and completely described below with reference to the accompanying drawings of the embodiments of the present disclosure. It is obvious that the described embodiments are merely a part of the embodiments of the present disclosure, rather than all of the embodiments. Based on the embodiments of the present disclosure, all other embodiments obtained by those of ordinary skill in the art without inventive work shall fall within the scope of protection of the present disclosure.

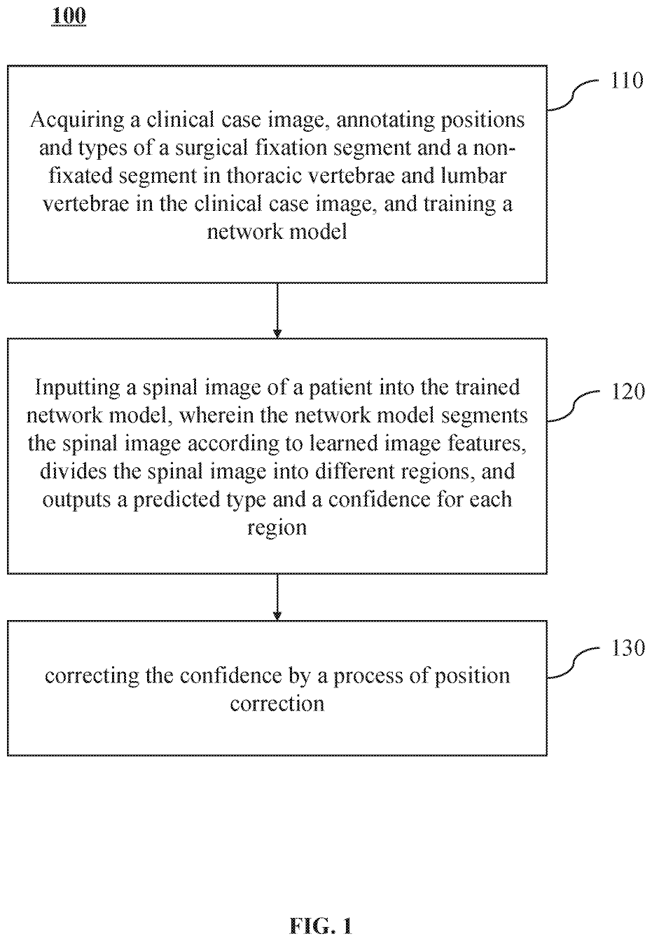

FIG. 1 is a flowchart of an exemplary process for surgical segment selection in AIS assisted by deep learning according to some embodiments of the present disclosure.

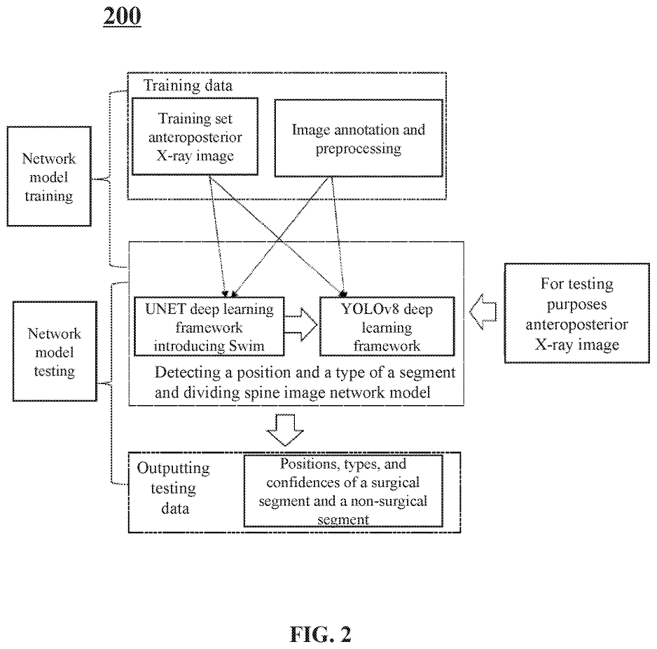

FIG. 2 is an exemplary schematic diagram of an exemplary process for surgical segment selection in AIS assisted by deep learning according to some embodiments of the present disclosure.

In some embodiments, as shown in FIG. 1, a process 100 may be performed by a processor. The process 100 may include step 110 to step 130.

In some embodiments, the processor may include a Central Processing Unit (CPU), an Application-Specific Integrated Circuit (ASIC), an Application-Specific Instruction-Set Processor (ASIP), a Physical Processing Unit (PPU), a Digital Signal Processor (DSP), a processor, a microprocessor unit, a Reduced Instruction Set Computer (RISC), a microprocessor, or any combination thereof. In some embodiments, the processor may be local or remote. In some embodiments, the processor may be implemented on a cloud platform. Merely by way of example, the cloud platform may include a private cloud, a public cloud, a hybrid cloud, a community cloud, a distributed cloud, an internal cloud, a multi-tier cloud, or the like, or any combination thereof.

Step 110, acquiring a clinical case image, annotating positions and types of a surgical fixation segment and a non-fixated segment in thoracic vertebrae and lumbar vertebrae in the clinical case image, and training a network model.

The clinical case image refers to a digital image showing spinal morphology of an AIS postoperative follow-up patient. For example, the clinical case image may be a standing whole-spine anteroposterior X-ray digital image of a patient with AIS.

In some embodiments, the processor may acquire clinical case images in a plurality of manners.

For example, the processor may screen a whole-spine anteroposterior X-ray image of an AIS postoperative follow-up patient that meet acquisition criteria from a medical record system of a hospital (such as a PACS system), and after quality control and anonymization processing, use the whole-spine anteroposterior X-ray image of the AIS postoperative follow-up patient as clinical case image.

For example, the processor may further directly obtain the whole-spine anteroposterior X-ray image of the AIS postoperative follow-up patient from a public spinal image dataset (such as SASIG), and use the whole-spine anteroposterior X-ray image of AIS postoperative follow-up patient as clinical case image.

In some embodiments, the acquisition criteria for the clinical case image include: (i) diagnosing the patient as an AIS patient; (ii) the patient undergoing posterior spinal correction with internal fixation and bone graft fusion; (iii) a follow-up period of the patient exceeding 2 years; (iv) a final follow-up clinical X-ray image of the patient indicating a good prognosis; and the exclusion criteria include: (i) an age of the patient being less than 11 years or exceeding 18 years; (ii) the patient having a history of spinal surgery; (iii) the patient having a leg length discrepancy; (iv) the patient having a spinal fracture or a severe spinal tumor.

The acquisition criteria refer to rules and criteria for screening the clinical case image used for training a network model.

In some embodiments, the processor may diagnose the patient as an AIS patient in a plurality of manners.

For example, the processor may obtain medical records of the patient from the medical record system of the hospital, and determine patients with AIS conditions in the medical records as AIS patients. The present disclosure is not limited to the manner for diagnosing the patient as an AIS patient.

The follow-up period refers to a postoperative observation time interval from a date when an AIS patient undergoes the posterior spinal correction with internal fixation and bone graft fusion to a date when the AIS patient takes an X-ray image at a final follow-up. The final follow-up refers to the last follow-up of the AIS patient before the current date.

The good prognosis refers to a comprehensive evaluation result that an AIS patient has a satisfactory spinal correction effect (e.g., angle loss of scoliosis correction is less than 10°), a stable internal fixation position, no severe complications, and a good functional state of the patient at a final follow-up (>2 years) after undergoing posterior correction and fusion surgery.

In some embodiments of the present disclosure, acquiring clinical case images through the acquisition criteria can ensure high quality, homogeneity, and clinical reliability of training data through strict case screening criteria, provide an accurate ‘gold standard’ annotation basis for a deep learning model, thereby improving prediction accuracy and clinical applicability of the surgical segment selection.

The surgical fixation segment refers to a vertebral segment that needs to implant internal fixation devices (such as screws, hooks, or rods, or the like) and undergo bone graft fusion in spinal fusion surgery.

The non-fixated segment refers to a vertebral segment that does not implant internal fixation devices and retains mobility in spinal fusion surgery.

The position refers to a pixel coordinate range. For example, a position of the surgical fixation segment may be a pixel coordinate range of a vertebral segment that needs to implant internal fixation devices in the clinical case image, and a position of the non-fixated segment may be a pixel coordinate range of a vertebral segment that does not implant internal fixation devices and retains mobility in the clinical case image.

The type refers to a classification type of a vertebral segment. For example, the type may include a surgical fixation segment and a non-fixated segment. For example, if the type is normalized into a digital form, the type may include 0 (surgical fixation segment) and 1 (non-fixated segment).

In some embodiments, the processor may annotate positions and types of the surgical fixation segment and the non-fixated segment in thoracic vertebrae and lumbar vertebrae in the clinical case image in a plurality of manners.

For example, the processor may read annotation information of the clinical case image by a doctor from the medical record system, and annotate the annotation information on the clinical case image.

In some embodiments, the processor may import the acquired clinical case image into LabelMe, output a JavaScript Object Notation (JSON) file through the LabelMe, read the annotation points of the clinical case image in the JSON file, convert the annotation points according to the relative positions of the annotation points relative to an entire image of the clinical case image to obtain the relative positions of points of the surgical fixation segment and the non-fixated segment, store the relative positions of points and the type information into a text file, and use the text file as a training sample input for a deep learning network model.

The annotation point refers to a two-dimensional pixel coordinate point used for identifying a spatial position of the vertebral body.

In some embodiments, the processor may determine the annotation points based on the positions of the surgical fixation segment and the non-fixated segment. For example, the processor may determine pixel geometric centers of the surgical fixation segment and the non-fixated segment based on the pixel coordinate ranges of the surgical fixation segment and the non-fixated segment, and use pixel coordinates corresponding to the pixel geometric centers as the annotation points of the surgical fixation segment and the non-fixated segment.

The relative position refers to the position of the annotation point relative to the clinical case image. For example, if a lower left corner of the clinical case image is a coordinate origin, the relative position may be position coordinates of the annotation point under the coordinate system. The relative position may be expressed as (x, y), wherein x is an abscissa of the annotation point under the coordinate system, and y is an ordinate of the annotation point under the coordinate system.

The relative position of point refers to a relative coordinate range of the surgical fixation segment and the non-fixated segment in the clinical case image. The relative position of point can be expressed as (xₘₐₓ~xₘᵢₙ, yₘₐₓ~yₘᵢₙ), wherein xₘₐₓ is a maximum abscissa of the surgical fixation segment or the non-fixated segment under the coordinate system, xₘᵢₙ is a minimum abscissa of the surgical fixation segment or the non-fixated segment under the coordinate system, yₘₐₓ is a maximum ordinate of the surgical fixation segment or the non-fixated segment under the coordinate system, and yₘᵢₙ is a minimum ordinate of the surgical fixation segment or the non-fixated segment under the coordinate system.

In some embodiments, the processor may convert positions of the surgical fixation segment and the non-fixated segment into the relative positions of points of the surgical fixation segment and the non-fixated segment in a plurality of manners.

For example, for the surgical fixation segment and the non-fixated segment, the processor may determine a scaling factor when the annotation points are converted into the relative positions, multiply the scaling factor by the pixel coordinate range, and obtain the relative positions of points.

The text file refers to a file storing the relative positions of points and the types of the surgical fixation segment and the non-fixated segment. For example, the text file may be in a txt format.

In some embodiments of the present disclosure, through LabelMe annotation, coordinate normalization, and format conversion, the original clinical case images are converted into standardized deep learning training samples, which realizes unified data format, size-independent processing, and model input adaptation, and provides a high-quality data basis for subsequent network training and the confidence correction.

Step 120, inputting a spinal image of a patient into the trained network model, wherein the network model segments the spinal image according to learned image features, divides the spinal image into different regions, and outputs a predicted type and a confidence for each region.

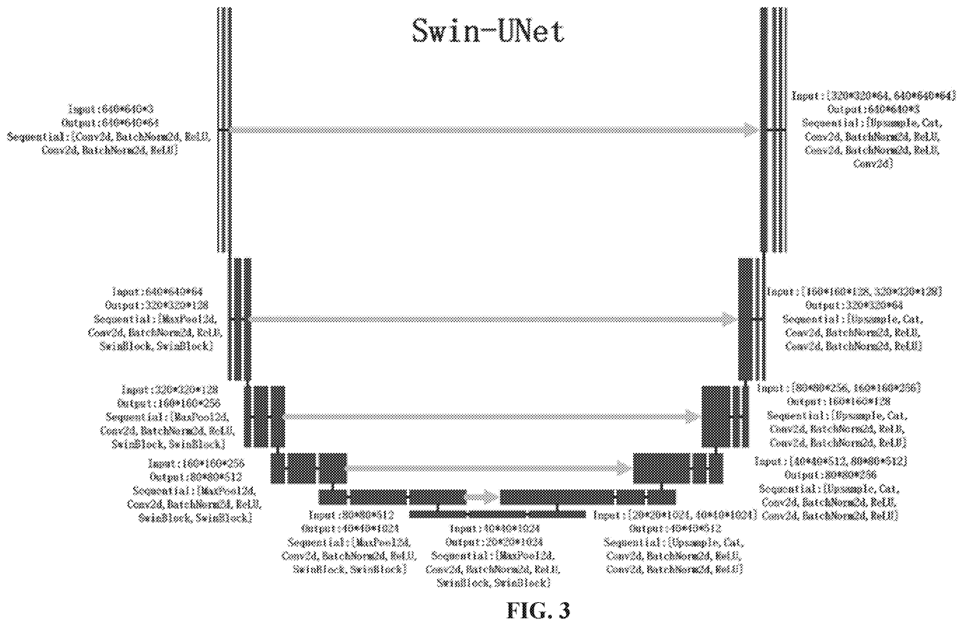

FIG. 3 is a diagram of an exemplary structure of Swin-UNet according to some embodiments of the present disclosure.

The spinal image refers to a digital image showing the preoperative spinal morphology of an AIS patient. In some embodiments, the spinal image and the clinical case image are both digital images depicting the preoperative spinal morphology of the AIS patient. The AIS patient corresponding to the spinal image has not yet undergone surgery, whereas the patient corresponding to the clinical case image has already undergone surgery. The clinical case image is clinical history data of the AIS patient.

In some embodiments, the processor may obtain the spinal image in a plurality of manners.

For example, the processor may receive a digital image of preoperative spinal morphology uploaded by a doctor to a clinical case system, and use the digital image as the spinal image.

The network model refers to a model for predicting a type of each region of the spinal image. In some embodiments, the network model may be a machine learning model. For example, the network model may be a Graph Neural Network (GNN).

In some embodiments, the network model employs a process combining a UNet learning framework and a YOLOv8 learning framework, inputs an annotated spinal image into the network model, and after training iterations, the trained network model automatically recognizes image features of the spinal image according to a deep learning result of the spinal image, segments the spinal image based on the image features, divides the spinal image into the different regions, and outputs a predicted type and the confidence for each region.

In some embodiments, the UNet learning framework includes a Swin Transformer module.

The Swin Transformer module refers to a visual Transformer architecture unit based on a shifted window (Shifted Window) mechanism. In some embodiments, the Swin Transformer module may replace a traditional convolutional block to enhance semantic segmentation capability of the spinal image through global-local adaptive feature extraction.

In some embodiments of the present disclosure, introducing the Swin Transformer module into the UNet learning framework improves a feature extraction capability of the UNet learning framework.

In some embodiments of the present disclosure, by constructing the network model through a dual-framework combination of the UNet learning framework and the YOLOv8 learning framework, the synergy of coarse-grained semantic segmentation and fine-grained instance detection for spinal images can be realized, and the accuracy, completeness, and clinical utility of surgical segment recognition may be improved.

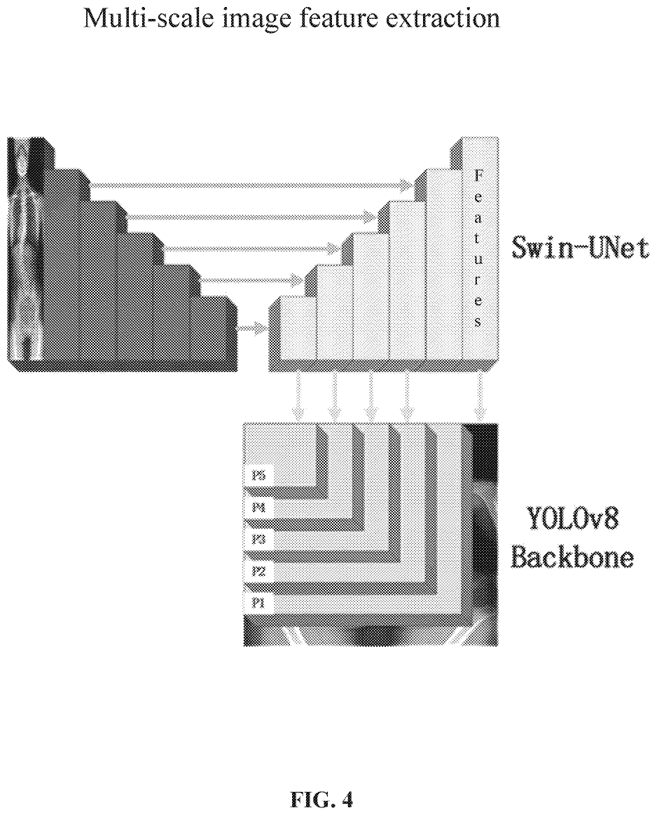

FIG. 4 is a schematic diagram of an exemplary multi-scale image feature extraction according to some embodiments of the present disclosure.

In some embodiments, as shown in FIG. 4, the processor may concatenate a coarse segmentation result output by the UNet learning framework with the original spinal image and input the concatenated image into a P1 layer of the YOLOv8 learning framework, extract initial multi-scale features of the concatenated spinal image, concatenate the initial multi-scale features with inputs of P2, P3, P4, and P5 layers of the YOLOv8 learning framework, and then use a FPN structure of the YOLOv8 learning framework to further extract the multi-scale features of the concatenated spinal image.

The initial multi-scale features refer to a set of feature representations extracted by the P1 layer of the YOLOv8 learning framework from the spinal image, having different spatial resolutions and receptive field (Receptive Field) sizes.

The inputs of the P2, P3, P4, and P5 layers of the YOLOv8 learning framework refer to downsampling of an output of a previous layer. For example, an input of the P2 layer of the YOLOv8 learning framework is downsampling of the initial multi-scale features output by the P1 layer of the YOLOv8 learning framework; an input of the P3 layer of the YOLOv8 learning framework is downsampling of the multi-scale features output by the P2 layer of the YOLOv8 learning framework; an input of the P4 layer of the YOLOv8 learning framework is downsampling of the multi-scale features output by the P3 layer of the YOLOv8 learning framework; and an input of the P5 layer of the YOLOv8 learning framework is downsampling of the multi-scale features output by the P4 layer of the YOLOv8 learning framework.

The multi-scale features refer to a set of feature representations extracted by a FPN structure of the YOLOv8 learning framework, having different spatial resolutions and receptive field (Receptive Field) sizes.

In some embodiments, the processor may determine the multi-scale features in a plurality of manners.

For example, the processor concatenates the coarse segmentation result output by the UNet learning framework with the spinal image by channel, and inputs the concatenated image into the P1 layer of the YOLOv8 learning framework. The P1 layer extracts the initial multi-scale features through a convolution operation. These initial multi-scale features are sequentially passed to the P2, P3, P4, and P5 layers via convolutional downsampling of the YOLOv8 backbone network. Specifically, the P1 layer retains full-scale features with the highest resolution to locate small vertebral bodies; the P2 layer reduces the resolution by half through a convolution with a stride of 2 and fuses high-resolution features passed from the P1 layer; the P3 layer continues to downsample to one-fourth scale of the original image to capture medium-sized vertebral body structures; the P4 layer is further compressed to one-eighth scale to perceive the context of vertebral body cluster regions; and the P5 layer finally reduces to one-sixteenth scale to obtain a global curvature morphology of the entire spine. Features of each layer are horizontally connected from top to bottom via the FPN, upsampling deep semantic information layer by layer and fusing it with shallow localization features, thereby forming complete multi-scale features that simultaneously include high-resolution details and low-resolution semantics.

According to some embodiments of the present disclosure, through the YOLOv8 learning framework, early fusion of semantic segmentation features and original visual information is achieved, allowing the network to obtain prior guidance for the spinal region at the highest resolution stage and effectively improving the targetedness of subsequent feature extraction. Deep global semantic information and shallow high-resolution localization features fully interact. The ultimately output multi-scale features simultaneously possess fine-grained capability for accurate localization of individual vertebral bodies and context understanding capability for the overall spinal curvature morphology. This significantly improves the accuracy and robustness of the surgical fixation segment and non-fixated segment recognition, especially exhibiting good generalization performance for AIS cases of different body types and different curvature types.

In some embodiments, an input of the network model is the spinal image, and an output the predicted type and confidence corresponding to the predicted type.

In some embodiments, the network model is obtained by training based on a plurality of sets of first training samples with first training labels. A set of first training samples may include sample spinal images. The first training labels corresponding to a set of first training samples may be sample predicted types.

In some embodiments, the processor may use clinical case images from historical cases that have been classified and undergone surgery as the first training samples, and determine a type corresponding to the clinical case images as the first training labels.

In some embodiments, the processor may perform a plurality of rounds of iterative training on the initial network model based on the plurality of sets of first training samples with the first training labels, until a training end condition is met, to obtain the trained network model. At least one round of iterative training includes: inputting a set or the plurality of sets of first training samples into the initial network model; obtaining model outputs corresponding to the set or the plurality of sets of first training samples; substituting the model outputs corresponding to the set or the plurality of sets of first training samples, and the first training labels corresponding to the set or the plurality of sets of first training samples, into a formula of a pre-defined loss function to determine a value of the loss function; iteratively updating model parameters in the initial network model according to the value of the loss function; and ending the iteration until the training end condition is met, to obtain the trained network model. Iterative updating of the initial network model parameters may be performed in a plurality of manners. For example, the parameters may be updated based on a gradient descent manner. The training end condition may include convergence of the loss function, or a number of iterations reaching an iteration threshold, or the like. The training end condition may be convergence of the loss function, a number of iterations reaching a preset iteration threshold, or the value of the loss function being less than a preset function value threshold, or the like.

The image feature refers to a digital information representation extracted from an anteroposterior radiograph of the spine that characterizes an anatomical structure and a pathological morphology of the vertebral body. For example, the image feature may include multi-scale features.

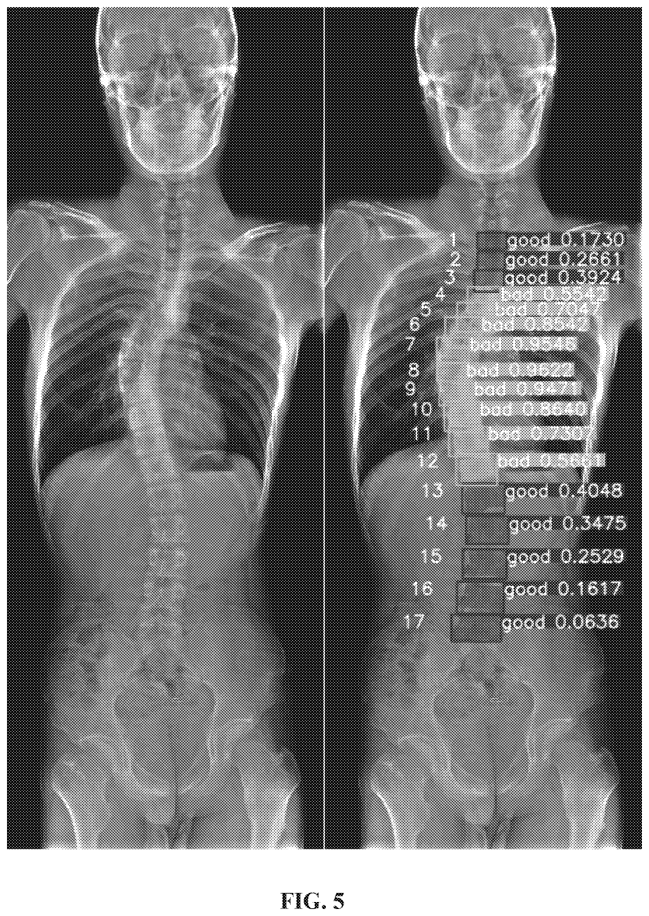

FIG. 5 is a display diagram of an exemplary spinal instance segmentation result according to some embodiments of the present disclosure.

In some embodiments, the processor may segment the image into a plurality of manners based on learned image features. The segmentation results may be shown in FIG. 5.

For example, the processor may, based on multi-scale instance features extracted by the YOLOv8 learning framework, perform instance-level segmentation on the image through cross-layer fusion of the FPN and detection head classification, to locate positions of each vertebral body and assign surgical fixation or non-fixated type labels, thereby achieving multi-level image parsing from region segmentation to individual identification.

As another example, the processor may perform, based on coarse-grained semantic features extracted by the UNet learning framework, pixel-level segmentation on the spinal image through upsampling recovery and skip connection fusion of an encoder-decoder structure, to distinguish between a spinal region and a background.

As a further example, the processor may further perform, based on global context features extracted by the Swin Transformer module, hierarchical segmentation on the image through long-range dependency modeling of the shifted window self-attention mechanism, to capture structural correlations between vertebral bodies.

The predicted type refers to a pixel region output by the network model belonging to a surgical fixation segment type or a non-fixated segment type.

In some embodiments, in the calculating the confidence of each vertebral body classification by the network model, the processor calculates the confidence of each vertebral body classification using the YOLOv8 learning framework.

The vertebral body classification refers to a classification of surgical fixation segments and non-fixated segments in thoracic vertebrae and lumbar vertebrae within the spinal image.

Step 130, correcting the confidence by a process of position correction.

The process of position correction by automatic calculation and manual correction refers to a manner for correcting the confidence. For example, the process of position correction by automatic calculation and manual correction may include automatic calculation and manual correction.

In some embodiments, the processor may correct the confidence in a plurality of manners.

For example, the processor may perform the correcting the confidence by using the process of ‘automatic calculation and manual correction’, the automatic calculation portion may determine the confidence for each vertebral body classification by the network model, and then a technician may manually correct the confidence for each vertebral body classification based on historical experience.

In some embodiments, the correcting the confidence includes: (1) determining, by the network model, the confidence for each vertebral body classification; (2) determining an offset of each vertebral body relative to a C7 vertical line (for a thoracic curve) or a center sacral vertical line (for a thoracolumbar curve or a lumbar curve), determining an apical vertebral body of a fusion curve, calculating a distance between each vertebral body and the apical vertebral body of the fusion curve, and using the distance as a distance weight for correcting the confidence; (3) correcting the confidence by the distance weight between each vertebral body and the apical vertebral body of the fusion curve, wherein when there are a plurality of apical vertebral bodies of fusion curves, a vertebral body weight between the apical vertebral bodies of the fusion curves is the same as the distance weight of the apical vertebral bodies of the fusion curves; (4) correcting the confidence according to prior knowledge, the distance weight, and the vertebral body weight, so that the confidence better conforms to clinical prior rules for fusion segment selection in AIS surgery.

The vertebral body classification refers to types of different vertebral bodies in the spinal image.

The apical vertebral body of fusion curve refers to a vertebral body with the largest distance from the central axis of the spine in a scoliosis fusion surgery. For example, the apical vertebral body of fusion curve may be the vertebral body with the largest offset relative to the C7 vertical line (for the thoracic curve) or the center sacral vertical line (for the thoracolumbar curve or the lumbar curve).

In some embodiments, the processor may determine the distance between the vertebral body and the apical vertebral body of the fusion curve in a plurality of manners.

For example, assuming the patient is a thoracic curve AIS patient, and T8 is determined to be the apical vertebral body of the fusion curve by calculation, the processor, taking T8 as a reference point, calculates a segment distance between each vertebral body and T8 towards the cranial side and the caudal side respectively, the distance between T7 and T8 is 1, the distance between T6 and T8 is 2, the distance between T5 and T8 is 3, the distance between T4 and T8 is 4, the distance between T3 and T8 is 5, and so on up to T1; and towards the caudal side, the distance between T9 and T8 is 1, the distance between T10 and T8 is 2, the distance between T11 and T8 is 3, the distance between T12 and T8 is 4, the distance between L1 and T8 is 5, and the distance between L2 and T8 is 6, and so on up to L5.

The distance weight refers to a confidence correction coefficient corresponding to the anatomical distance of each vertebral body relative to the apical vertebral body of the fusion curve.

In some embodiments of the present disclosure, the processor may determine the distance weight based on the distance between the vertebral body and the apical vertebral body of the fusion curve.

For example, the processor may determine that the closer the distance between the vertebral body and the apical vertebral body of the fusion curve is, the higher the weight is, and the farther the distance is, the lower the weight is.

Merely by way of example, the distance value may directly serve as a weight for confidence correction; T8 has a distance value of 0 corresponding to the highest weight; T6 and T10 have a distance value of 2 corresponding to the second highest weight; and T4 and T12 have a distance value of 4 corresponding to the third highest weight, thereby forming a weight distribution that decreases bilaterally with T8 as the center. When there are double primary curves and both T8 and L2 are apical vertebral bodies, the vertebral bodies between T8 and L2 (T9 to L1) are treated with the same weight as the apical vertebral bodies, that is, all the vertebral bodies in this range acquire the highest weight, thereby ensuring that the fusion range must cross the apical vertebral body regions of the two curves.

In some embodiments, the processor may multiply the distance weight of each vertebral body by the confidence of the predicted type corresponding to the vertebral body to obtain a corrected confidence.

In some embodiments, when there are a plurality of apical vertebral bodies of fusion curves, the distance weight of the vertebral bodies between the apical vertebral bodies of the fusion curves is the same as the distance weight of the apical vertebral bodies of the fusion curves.

Merely by way of example, the distance weight of the apical vertebral body of fusion curve is 1, and the farther the distance from the apical vertebral body of fusion curve is, the smaller the distance weight is. If there are a plurality of apical vertebral bodies of fusion curves, the distance weight of the vertebral bodies between the two apical vertebral bodies of fusion curves is the same as the distance weight of the apical vertebral bodies of fusion curves themselves, that is, the distance weights of the vertebral bodies between the two apical vertebral bodies of fusion curves are both 1.

In some embodiments of the present disclosure, the processor determines an initial confidence for each vertebral body classification by the network model, determines an apical vertebral body of the fusion curve based on the offset of the vertebral body relative to the C7 vertical line or the center sacral vertical line, and uses the segment distance between the vertebral body and the apical vertebral body as a weight for correcting the confidence, so that the corrected confidence distribution presents a form of high in the middle and low on both ends with the apical vertebral body as the center. At the same time, the processor also uses prior knowledge constraints to ensure that the fusion range must cross the apical vertebral body and avoid key vertebral bodies such as T1 and L5, thereby organically combining the probability prediction of deep learning with clinical surgical planning principles, reducing noise interference of the network model, improving the accuracy and interpretability of surgery segment selection, enabling clinicians to make personalized surgical decisions based on the confidence distribution conforming to clinical logic, and ultimately achieving a balance between the optimal spinal correction effect and the maximum preservation of spinal mobility.

In some embodiments, the processor may also generate at least one candidate fusion segment range for consecutive vertebral bodies of the surgical fixation segment and confidences corresponding to the consecutive vertebral bodies according to the predicted type; construct a spinal structure model according to vertebral body boundary information corresponding to the spinal image, determining a postoperative fixation area based on the at least one candidate fusion segment range, applying a structural label and a connection constraint corresponding to the postoperative fixation area to the spinal structure model, and calculate a postoperative compensation parameter of the vertebral body outside the candidate fusion segment range; generate a compensation risk value according to the postoperative compensation parameter, and correct the confidence of the at least one candidate fusion segment range according to the compensation risk value to obtain a corrected confidence; and input the corrected confidence as feedback information into a training stage of the network model to update model parameters in the network model.

The consecutive vertebral bodies refer to a plurality of vertebral bodies with adjacent and uninterrupted segment numbers in the spinal image. For example, the processor may parse the spinal image, annotate segment numbers for each vertebral body from top to bottom, and determine a plurality of vertebral bodies with adjacent and uninterrupted segment numbers as the consecutive vertebral bodies.

The candidate fusion segment range refers to a range composed of one or more candidate consecutive surgical fixation segments formed according to the anatomical sequence. The anatomical sequence refers to a sequence of vertebral bodies arranged successively from a cranial side to a caudal side according to a natural anatomical structure and a physiological curvature of the human spine. In some embodiments, the candidate fusion segment range may be characterized by an upper vertebral body number and a lower vertebral body number. For example, T10 to L2 indicates a consecutive fusion segment from the 10th thoracic vertebra (T10) to the 2nd lumbar vertebra (L2).

In some embodiments, the processor forms, based on the positions, types, and corresponding confidences of each vertebral body segment output by the network model and in combination with the confidence correction result, one or more candidate consecutive fusion intervals according to the anatomical sequence after evaluating the possibility of each vertebral body being included in a surgical fixation/fusion range.

For example, according to the anatomical sequence of vertebral bodies from top to bottom, the processor may sort the positions and types of different vertebral body segments output by the network model. The processor may then read the confidence corresponding to each vertebral body and screen out a vertebral body recommended to be included in a surgical fixation/fusion range based on the confidence. The vertebral body with relatively high corrected confidence may be regarded as prioritized vertebral body for inclusion in the surgical fixation/fusion range. The vertebral body with relatively low corrected confidence may be regarded as deprioritized or excluded vertebral body. If adjacent vertebral bodies all satisfy preset inclusion conditions, they may be merged into the same consecutive interval. If a certain vertebral body does not satisfy the inclusion conditions, the consecutive interval may be segmented at this point.

For each consecutive interval, an overall interval score may be determined based on the confidence of each vertebral body within the interval (an average value, a weighted average value, a minimum value, or a cumulative sum, or the like, may be adopted). According to the interval scores sorted from high to low, one or more candidate fusion segment ranges may be screened out, wherein each range is characterized by an upper vertebral body number and a lower vertebral body number (for example, T10 to L2).

In some embodiments, the postoperative compensation parameter comprises at least one of the predicted residual lateral inclination angle and the central offset distance of the vertebral body outside the candidate fusion segment range.

The predicted residual lateral inclination angle refers to a parameter obtained by predicting a coronal plane inclination angle that may still remain for the vertebral body outside the candidate fusion segment range after the candidate fusion segment range is determined. In some embodiments, the predicted residual lateral inclination angle may be used to reflect a degree of residual vertebral deformity.

The central offset distance refers to a deviation distance of a vertebral body center point, a segment centerline, or an overall central axis of a compensation area outside the candidate fusion segment range relative to a target reference line. In some embodiments, the central offset distance may be used to reflect a postoperative trunk balance state.

In some embodiments of the present disclosure, by specifically defining the postoperative compensation parameter as at least one of the predicted residual lateral inclination angle and the central offset distance, the influence of the candidate fusion segment range on residual deformity and overall balance may be converted into calculable indicators, which facilitates risk quantification and the confidence correction.

In some embodiments, the processor may determine the postoperative compensation parameter in a plurality of manners.

For example, the processor may determine the postoperative compensation parameter based on the spinal structure model through the graph neural network.

An input of the graph neural network includes the spinal structure model, and an output includes the postoperative compensation parameter.

Merely by way of example, the processor can train the graph neural network based on a plurality of sets of second training samples with second training labels. The second training samples may be the spinal structure models corresponding to the clinical case images in historical data. The second training labels corresponding to the second training samples may be the postoperative compensation parameters corresponding to the clinical case images in historical data. The processor performs multi-round iterative training on an initial graph neural network based on a plurality of sets of second training samples with second training labels, and terminate the training when the iteration termination condition is satisfied, to obtain a trained graph neural network. At least one round of iterative training includes: inputting one or more sets of second training samples into the initial graph neural network to obtain model output corresponding to the one or more sets of second training samples; substituting the model output corresponding to the one or more sets of second training samples and the second training labels corresponding to the one or more sets of second training samples into a formula of the pre-defined loss function to determine the value of the loss function; and iteratively updating the model parameters in the initial graph neural network according to the value of the loss function, and terminating the iteration when the iteration termination condition is satisfied, to obtain a trained graph neural network. The iterative updating of the model parameters of the initial graph neural network may be performed in a plurality of manners. For example, the iterative updating may be performed based on a gradient descent manner. The iteration termination condition includes convergence of the loss function or iteration times reaching an iteration times threshold, or the like. The iteration termination condition may be convergence of the loss function, iteration times reaching a preset times threshold, or the value of the loss function being less than a preset function value threshold, or the like.

The compensation risk value refers to an evaluation value used to reflect a degree of risk of a certain candidate fusion segment range causing postoperative imbalance, residual curvature, or under-compensation.

In some embodiments, the processor may determine the compensation risk value in a plurality of manners.

For example, for selected vertebral bodies in a compensation area, the processor may score the selected vertebral bodies based on the predicted residual lateral inclination angle and the central offset distance, by comparing against clinically acceptable thresholds (e.g., inclination ≤10° and offset ≤20 mm for safety). The more the threshold is exceeded, the higher the risk score becomes (e.g., 0-1 point). Local risks of the selected compensation vertebral bodies may be merged (usually taking a maximum value (focusing on the most dangerous segment) or an average value (reflecting an overall compensation level)). The merged risk value of compensation vertebral bodies may be used as a compensation risk value for a candidate fusion segment.

The corrected confidence refers to the confidence after correction.

In some embodiments, the processor may correct the confidence of the candidate fusion segment range based on the postoperative compensation parameter.

For example, the processor can evaluate the confidence of the fixation segments (i.e., the vertebral bodies within the candidate fusion segment range) based on the postoperative compensation risk value. The higher the risk, the lower the new confidence becomes (e.g., corrected confidence=the confidence×(1−the compensation risk value)).

In some embodiments, the processor may update the model parameters of the network model in a plurality of manners.

For example, in a standard training stage, in addition to receiving original annotations (whether each vertebral body is a surgical fixation segment) to determine cross-entropy classification loss, the network model also uses mean square error or Kullback-Leibler (KL) divergence between the confidence obtained by forward inference on the spinal image and the corrected confidence (a soft label) generated by fusion with the postoperative compensation risk value as confidence matching loss. The network model determines a total loss based on the cross-entropy classification loss and the confidence matching loss (Total loss=classification loss+λ×confidence matching loss, where λ is a default preset hyperparameter). The network model passes the total loss back to the network through a backpropagation algorithm, automatically adjusting weights and biases, so that the output confidence of the network model is closer to the corrected confidence during the next prediction. Meanwhile, the network model stops updating the model parameters when the network model converges.

In some embodiments of the present disclosure, by introducing a postoperative compensation state determination and feedback update mechanism after initial network classification results, simple image recognition results can be further constrained to the level of postoperative balance and fusion rationality, thereby enhancing clinical consistency of the fusion segment selection and the stability of the model training direction.

In some embodiments, the processor may also output the planning result of the target fusion segment based on the corrected confidence; and based on the planning result of the target fusion segment, generate the planning parameter set for display, storage, or invocation on the preoperative planning interface.

The planning result of the target fusion segment refers to a fusion segment range finally selected based on an updated confidence. For example, the planning result of the target fusion segment may be manifested as a combined result of an upper fusion vertebral body, a lower fusion vertebral body, and an intermediate consecutive fixation segment.

In some embodiments, the processor may output the planning result of the target fusion segment in a plurality of manners.

For example, the processor may sort all candidate fusion segment ranges from high to low according to the corresponding corrected confidence. The processor may select one or a plurality of candidate fusion segment ranges with the best ranking as the planning result of the target fusion segment. If a difference in the corrected confidence between a first-ranked candidate fusion segment range and a second-ranked candidate fusion segment range is lower than a preset threshold, then the first-ranked candidate fusion segment range and the second-ranked candidate fusion segment range are simultaneously output as the planning result of the target fusion segment range. If the difference in the corrected confidence between the first-ranked candidate fusion segment range and the second-ranked candidate fusion segment range is higher than or equal to the preset threshold, then the first-ranked candidate fusion segment range is output as the planning result of the target fusion segment range. The preset threshold refers to a parameter used to determine whether to simultaneously output two candidate fusion segment ranges as the planning result of the target fusion segment range. In some embodiments, the preset threshold may be preset by a skilled person based on experience.

The preoperative planning interface refers to an electronic interface that presents surgical planning to a doctor.

The planning parameter set refers to a parameter set for display, storage, or invocation on the preoperative planning interface. In some embodiments, the planning parameter set may be used to characterize risk reminder information related to the target fusion segment. For example, a preoperative planning parameter set includes, but is not limited to, at least one of the upper fusion boundary, the lower fusion boundary, the target correction range, the candidate key observation vertebral bodies, and the risk reminder items.

The upper fusion boundary and the lower fusion boundary refer to the vertebral bodies that are included in a fixation area and are the uppermost (i.e., closest to a head) or the lowermost (i.e., closest to a pelvis) vertebral bodies in a spinal fusion surgery.

The target correction range refers to a range of curved vertebral bodies that need to be corrected.

The candidate key observation vertebral bodies may be vertebral bodies adjacent to a fixation area or vertebral bodies with abnormal compensation parameters.

The risk reminder items may be a compensation risk level, adjacent segment degeneration risk, or the like.

In some embodiments, the processor may determine the planning parameter set in a plurality of manners based on the planning result of the target fusion segment.

For example, the processor may extract an upper fusion vertebral body number and a lower fusion vertebral body number from the planning result of the target fusion segment. The processor may determine an apical vertebral body position, a vertebral body range involved in a fusion curve, and a Cobb angle based on preoperative image measurement. The processor may invoke a pre-calculated compensation risk value and an updated confidence level, and generate a preoperative planning parameter set in combination with a clinical database or a preset rule.

The preoperative planning parameter set may be output to the preoperative planning interface for visual display, or may be stored in clinical case data.

In some embodiments of the present disclosure, continuing to output the planning result of the target fusion segment and the preoperative planning parameter set after completion of the confidence update may enable a model result to be further transformed from ‘classification determination’ into ‘planning output available for preoperative use’, thereby improving clinical applicability and operability of the result.

In some embodiments, generating the compensation risk value according to the postoperative compensation parameter and correcting the confidence of the candidate fusion segment range based on the compensation risk value may further include introducing the spatial posture compensation mechanism. The spatial posture compensation mechanism includes: extracting an additional feature characterizing a spatial posture of a vertebral body based on the local image region corresponding to each vertebral body in the spinal image, and determining the rotation state information of each vertebral body according to the additional feature. The processor may also perform multi-dimensional feature fusion or weighted summation on the rotation state information and the compensation risk value to generate the target update factor, and correct the confidence according to the target update factor.

The spatial posture compensation mechanism refers to an internal adjustment manner in which, when a certain vertebral body segment is classified as a surgical fixation area (fusion segment), in order to maintain balance and stability of an overall spinal sequence, the remaining non-fixated segments (compensation areas) actively or passively adjust their geometric morphology, spatial positions, and relative angles to compensate for functional defects and morphological changes caused by the fixation area.

The additional feature refers to feature information that, in addition to a segmentation contour and classification confidence, is further used to characterize a spatial posture of a vertebral body. In some embodiments, the additional feature mainly reflects left-right asymmetry of a vertebral body, projection skew, and rotation tendency.

In some embodiments, the processor may determine the additional feature in a plurality of manners.

For example, the processor may identify positions of left and right corresponding local anatomical landmarks in the local image region of the vertebral body. The processor may extract, according to the positions of the local anatomical landmarks, correlation information features characterizing left-right differences and convert the correlation information features into vectorized features. The processor may use the vectorized features as the additional feature.

The positions of local anatomical landmarks refers to local key point coordinate position that can reflect anatomical structure features of the vertebral body and has clear anatomical significance.

In some embodiments, the processor may segment the spinal image using a backbone network (such as UNet+YOLOv8), and extract an independent image block of each vertebral body (i.e., a local image region). For each vertebral body image block, the processor may perform inference using a specialized pedicle key point detection sub-network (e.g., HRNet) and identify and output pixel coordinates of the center point, the upper edge point, and the lower edge point of the left pedicle and the right pedicle, totaling 6 key points. The 6 key point coordinates of each vertebral body then constitute the local anatomical landmark position of the vertebral body, which is used as an input for a subsequent asymmetry feature extraction module for calculating vertebral body rotation-related features.

The correlation information features include at least one of position feature, size feature, and shape feature.

In some embodiments, the processor may extract position, size, and shape features by calculation based on the 6 key point coordinates of the left and right pedicles (left center, left upper, left lower; right center, right upper, right lower), and combine them into vectorized features.

In some embodiments, the processor may concatenate all correlation information features in a fixed order to form a multi-dimensional feature vector, wherein the multi-dimensional feature vector is the vectorized feature.

In some embodiments, the processor may identify positions of left and right corresponding local anatomical landmarks in the local image region of the vertebral body; extract, according to the positions of the local anatomical landmarks, correlation information features characterizing left-right differences and convert the correlation information features into vectorized feature.

The left-right differences refer to a geometrical relationship based on the local anatomical landmark positions (6 key points) on left and right sides of the same vertebral body. The left-right differences mainly reflect rotation tendency and the spatial posture of the vertebral body in a coronal plane projection.

In some embodiments, the correlation information features characterizing left-right differences include at least one of position feature, size feature, and shape feature.

The position feature may include at least one of a horizontal distance and a vertical distance between the left and right pedicles.

The size feature may include an area difference of corresponding regions of the left and right pedicles.

The shape feature may include a contour difference of corresponding regions of the left and right pedicles.

In some embodiments of the present disclosure, by further refining left-right difference features into three specific types of features: position, size, and shape, the granularity of the rotation state characterization may be improved, making the additional feature easier to quantify, combine, and reuse, and enhancing the fineness of the subsequent posture determination and the confidence update.

In some embodiments of the present disclosure, by extracting the rotation-related additional features based on the left and right corresponding local anatomical landmarks, the vertebral body posture determination may be enabled to be established on a clearer anatomical correspondence, thereby improving interpretability, stability, and subsequent fusion calculation accuracy of posture feature extraction.

The local image region refers to an independent image block corresponding to a single vertebral body extracted from the complete spinal image.

In some embodiments, the processor may determine a minimum bounding rectangle of each vertebral body as the local image region based on a segmentation result of the spinal image.

The rotation state information refers to a characterization result of a rotation degree, a rotation direction, or a rotation angle of a vertebral body reflected in a coronal plane projection (Coronal Plane).

In some embodiments, the processor may determine the rotation state information in a plurality of manners.

For example, the processor may determine the rotation state information through a neural network based on features after concatenation of key point coordinates, multi-scale image features, and vectorized features.

In some embodiments, the processor may extract multi-scale image features corresponding to each vertebral body output by the feature pyramid network of the YOLOv8 learning framework in the network model. The processor may concatenate and fuse the vectorized features with the multi-scale image features. The processor may input the concatenated and fused image features into a rotation angle regression head, and output, by the rotation angle regression head, a predicted vertebral rotation angle as the rotation state information of a corresponding vertebral body.

In some embodiments of the present disclosure, by fusing the vectorized features formed by anatomical differences with multi-scale image features output by the YOLOv8 feature pyramid, and by outputting the rotation state information by a rotation angle regression head, robustness of rotation determination and utilization efficiency of multi-source features may be enhanced.

The target update factor refers to an intermediate parameter obtained after fusing the rotation state information and the compensation risk value and used for performing joint correction on confidence.

In some embodiments, the processor may numerically normalize the rotation state information and the compensation risk value respectively; and obtain the target update factor by means of weighted summation. The weight is a preset weight, and the weight may be assigned based on clinical experience. For example, if clinical practice determines that the rotation information has a greater impact on surgical decision-making, then a weight of the rotation information is increased; if both are considered equally important, then 0.5 is assigned to each.

In some embodiments, the processor may determine the corrected confidence in a plurality of manners based on the target update factor. For example, the corrected confidence may be determined by the following formula: corrected confidence=confidence×(1−λ×target update factor), wherein λ is an adjustment coefficient (0<λ≤1), and λ may be preset based on clinical experience.

In some embodiments of the present disclosure, by introducing the spatial posture compensation mechanism during a confidence update process, a correction merely based on coronal plane geometrical relationships may be extended to a joint correction that simultaneously considers the vertebral body rotation information, thereby enhancing expression capability of AIS three-dimensional deformity features and improving planning reliability.

In some embodiments, the processor may perform the hard constraint on confidence corrected by the distance weight based on the clinical prior rules for scoliosis surgery.

The clinical prior rules refer to surgical segment selection rules used to limit a network output result. For example, the clinical prior rules may include that the target fusion segment needs to cross the apical vertebral body of a fusion curve, and exclude thoracic T1 and lumbar L5 vertebral bodies as options for the fusion segment.

The hard constraint refers to a constraint on a candidate fusion segment range that does not satisfy the clinical prior rules. For example, when a candidate fusion segment range does not satisfy the clinical prior rules, the hard constraint may be reducing the confidence of the candidate fusion segment range, excluding the candidate fusion segment range, or forcing correction of a boundary of the candidate fusion segment range.

In some embodiments of the present disclosure, the processor may perform the hard constraint on the corrected confidence in a plurality of manners.

For example, after completion of weight correction based on an apical vertebral body distance, the processor may check each candidate fusion segment range one by one to determine whether it satisfies the clinical prior rules. If a candidate fusion segment range does not cross an apical vertebral body, it is determined that the candidate fusion segment range does not satisfy the clinical prior rules, and the confidence of the candidate fusion segment range is significantly attenuated or directly set to zero. If a candidate fusion segment range includes thoracic T1 or lumbar L5 as a boundary candidate, then the corresponding candidate fusion segment range is excluded or adjusted to an adjacent acceptable segment.

In some embodiments of the present disclosure, by continuing to apply the hard constraint of the clinical prior rules after the distance weight correction, candidate segment ranges that obviously violate surgical principles may be further suppressed, making the confidence output more aligned with clinical decision-making habits, and reducing misjudgments caused by a model's sole reliance on image features.

For a person having ordinary skill in the art, the specific embodiments merely describe the present disclosure by way of example, and it is obvious that specific implementations of the present disclosure are not limited by the foregoing manners. Any non-substantive improvements made by adopting the inventive concept and technical solutions of the present disclosure, or the direct application of the inventive concept and technical solutions of the present disclosure to other occasions without improvements, shall fall within the protection scope of the present disclosure.

1. A method for surgical segment selection in adolescent idiopathic scoliosis (AIS) assisted by deep learning, comprising:
Step 1: segment labeling and preprocessing for spinal surgery: acquiring a clinical case image, annotating positions and types of a surgical fixation segment and a non-fixated segment in thoracic vertebrae and lumbar vertebrae in the clinical case image, and serves as inputs for a network model training;
Step 2: detecting the location and category of the segments, and segmenting a spinal image: inputting the spinal image of a patient into the trained network model, wherein the network model segments the spinal image according to learned image features, divides the spinal image into different regions, and outputs a predicted type and a confidence for each region; and
Step 3: confidence calibration: correcting the confidence by “automatic calculation and manual correction”.

2. The method for surgical segment selection in AIS assisted by deep learning according to claim 1, wherein acquisition criteria of the clinical case image include:
(i) diagnosing the patient as an AIS patient; (ii) the patient undergoing posterior spinal correction with internal fixation and bone graft fusion; (iii) a follow-up period of the patient exceeding 2 years; (iv) a final follow-up clinical image of the patient indicating a good prognosis; and exclusion criteria include: (i) an age of the patient being less than 11 years or exceeding 18 years; (ii) the patient having a history of spinal surgery; (iii) the patient having a leg length discrepancy; (iv) the patient having a spinal fracture or a severe spinal tumor.

3. The method for surgical segment selection in AIS assisted by deep learning according to claim 2, wherein the method further includes:
importing the acquired clinical case image data into LabelMe, outputting a JavaScript Object Notation (JSON) file generated by the LabelMe, reading annotation points of the clinical case image in the JSON file, performing conversion according to relative positions of the annotation points with respect to the clinical case image to obtain relative positions of points of the surgical fixation segment and the non-fixated segment, storing the relative positions of the points and the types into a text file, and using the text file as a training sample of the deep learning network model.

4. The method for surgical segment selection in AIS assisted by deep learning according to claim 1, wherein the network model uses a U-shaped Network (UNet) learning framework and a You Only Look Once version 8 (YOLOv8) learning framework, inputs the annotated spinal image into the network model, and after training iterations, the trained network model automatically recognizes image features of the spinal image according to the annotated spinal image, segments the spinal image based on the image features, divides the spinal image into different regions, and outputs a predicted type and a confidence for each region.

5. The method for surgical segment selection in AIS assisted by deep learning according to claim 4, wherein the UNet learning framework includes a Swin Transformer module.

6. The method for surgical segment selection in AIS assisted by deep learning according to claim 4, wherein a deep learning process of the network model includes: concatenating a coarse segmentation result output by the UNet learning framework with the spinal image and inputting a concatenated spinal image into a P1 layer of the YOLOv8 learning framework, extracting initial multi-scale features of the concatenated spinal image, concatenating the initial multi-scale features with inputs of P2, P3, P4, and P5 layers of the YOLOv8 learning framework, and using a feature pyramid network (FPN) structure of the YOLOv8 learning framework to further extract the multi-scale features of the concatenated spinal image.

7. The method for surgical segment selection in AIS assisted by deep learning according to claim 4, wherein the correcting the confidence includes:
(1) determining, by the network model, a confidence for each vertebral body classification;
(2) determining an offset of each vertebral body relative to a C7 vertical line (for a thoracic curve) or a center sacral vertical line (for a thoracolumbar curve or a lumbar curve), determining an apical vertebral body of a fusion curve, calculating a distance between each vertebral body and the apical vertebral body of the fusion curve, and using the distance as a distance weight for correcting the confidence;
(3) correcting the confidence by the distance weight between each vertebral body and the apical vertebral body of the fusion curve, wherein when there are a plurality of apical vertebral bodies of fusion curves, a vertebral body weight between the apical vertebral bodies of the fusion curves is the same as the distance weight of the apical vertebral bodies of the fusion curves; and
(4) correcting the confidence according to prior knowledge, the distance weight, and the vertebral body weight, so that the confidence better conforms to clinical prior rules for fusion segment selection in AIS surgery.

8. The method for surgical segment selection in AIS assisted by deep learning according to claim 7, wherein the correcting the confidence further includes:
generating at least one candidate fusion segment range for consecutive vertebral bodies of the surgical fixation segment and confidences corresponding to the consecutive vertebral bodies according to the predicted type;
constructing a spinal structure model according to vertebral body boundary information corresponding to the spinal image, determining a postoperative fixation area based on the at least one candidate fusion segment range, applying a structural label and a connection constraint corresponding to the postoperative fixation area to the spinal structure model, and calculating a postoperative compensation parameter of the vertebral body outside the candidate fusion segment range;
generating a compensation risk value according to the postoperative compensation parameter, and correcting the confidence of the at least one candidate fusion segment range according to the compensation risk value to obtain a corrected confidence; and
inputting the corrected confidence as feedback information into a training stage of the network model to update model parameters in the network model.

9. The method for surgical segment selection in AIS assisted by deep learning according to claim 8, wherein the postoperative compensation parameter includes at least one of a predicted residual lateral inclination angle and a central offset distance of the vertebral body outside the candidate fusion segment range.

10. The method for surgical segment selection in AIS assisted by deep learning according to claim 8, wherein after the correcting the confidence of the candidate fusion segment range according to the compensation risk value to obtain the corrected confidence, the method further includes:
outputting a planning result of a target fusion segment based on the corrected confidence; and
generating a planning parameter set for display, storage, or invocation on a preoperative planning interface based on the planning result of the target fusion segment.

11. The method for surgical segment selection in AIS assisted by deep learning according to claim 8, wherein the method further includes a spatial posture compensation mechanism, and the spatial posture compensation mechanism includes: extracting, based on a local image region corresponding to each vertebral body in the spinal image, an additional feature characterizing a spatial posture of each vertebral body, and determining rotation state information of each vertebral body according to the additional feature;
wherein the generating the compensation risk value according to the postoperative compensation parameter and correcting the confidence of the candidate fusion segment range according to the compensation risk value to obtain the corrected confidence includes:
performing multi-dimensional feature fusion or weighted summation on the rotation state information and the compensation risk value to generate a target update factor, and correcting the confidence according to the target update factor.

12. The method for surgical segment selection in AIS assisted by deep learning according to claim 11, wherein the extracting the additional feature characterizing the spatial posture of the vertebral body includes:
identifying positions of left and right corresponding local anatomical landmarks in the local image region of the vertebral body; and
extracting, according to the positions of the local anatomical landmarks, correlation information features characterizing left-right differences, and converting the correlation information features into vectorized features.

13. The method for surgical segment selection in AIS assisted by deep learning according to claim 12, wherein
the correlation information features characterizing the left-right differences include at least one of a position feature, a size feature, and a shape feature.

14. The method for surgical segment selection in AIS assisted by deep learning according to claim 12, in combination with the network model, wherein the determining the rotation state information of each vertebral body according to the additional feature includes:
extracting multi-scale image features corresponding to each vertebral body output by a feature pyramid network of the YOLOv8 learning framework in the network model;
concatenating and fusing the vectorized features with the multi-scale image features; and
inputting the concatenated and fused image features into a rotation angle regression head, and outputting, by the rotation angle regression head, a predicted vertebral rotation angle as the rotation state information of corresponding vertebral body.

15. The method for surgical segment selection in AIS assisted by deep learning according to claim 7, wherein in the confidence correction, the correcting the confidence according to the prior knowledge includes:
performing a hard constraint on the confidence corrected by the distance weight based on clinical prior rules for scoliosis surgery.

16. The method for surgical segment selection in AIS assisted by deep learning according to claim 7, wherein in the calculating the confidence of each vertebral body classification by the network model, the confidence of each vertebral body classification is calculated using the YOLOv8 learning framework.
